(19) United States
(12) Patent Application Publication
(10) Pub. No.: US 2015/0157553 A1
(43) Pub. Date: Jun. 11, 2015

(54) NON-CARIOGENIC, SUGAR-FREE CONFECTIONERY COMPOSITIONS CONTAINING ANTI-PLAQUE COMPONENTS AND NATURAL TREE, PLANTS AND TEA EXTRACTS FOR ORAL AND SYSTEMIC HEALTH BENEFITS

(71) Applicant: Kehinde A. Ayoola, Bowie, MD (US)

(72) Inventor: Kehinde A. Ayoola, Bowie, MD (US)

(21) Appl. No.: 14/100,394

(22) Filed: Dec. 9, 2013

(51) Int. Cl. A61K 8/97; A61Q 11/00; A23G 3/48; A61K 8/34

(52) U.S. Cl. CPC A61K 8/97; A61K 8/345; A61Q 11/00; A23G 3/48

(57) ABSTRACT

Non-cariogenic sugar-free confectionery compositions which deliver anti-plaque components to the mouth for oral health benefits and also deliver nutrients for systemic health benefits; wherein such confectionery compositions comprise of: (i) a safe and effective amount of natural tree, plants and tea extracts selected from moringa, matcha green tea, green tea, chia and mixtures thereof; (ii) one or more anti-plaque component or components; (iii) one or more natural, non-cariogenic, tooth-friendly sweetener or mixtures of sweeteners; (iv) a suitable confectionery carrier and (v) less than 10% water.

### BACKGROUND OF THE INVENTION

1. Field of the Invention

The present invention relates to non-cariogenic confectionery compositions which contain anti-plaque components to reduce the presence of dental plaque from the surfaces of the oral cavity, particularly, the teeth as in the case of chewable confectionery compositions. Furthermore, the invention relates to such confectionery compositions which contain effective amounts of natural plant, tree and tea extracts selected from moringa, matcha green tea, green tea, chia and mixtures thereof; extracts which contain many nutrients, anti-oxidants, minerals and other healthy components that confer additional oral health benefits and systemic health benefits.

2. The Prior Art

Dental plaque is a naturally occurring biofilm on teeth which consists of colonizing bacteria, bacterial byproducts and food debris. The accumulation of dental plaque is a major contributor to the development of dental caries and periodontal disease (gum disease) such as gingivitis and periodontitis. The challenge with plaque is that it adheres firmly to dental surfaces; it forms continuously; reforming even after an effective oral care regimen such as brushing, flossing.

Oral dentifrice such as toothpastes, gels, mouth washes are designed to serve as anti-plaque and anti-cavity compositions along with conferring other esthetic benefits such as teeth whitening. Although many oral care products are available to reduce plaque formation, the limitations of these products are that (i) their presence in the mouth and the duration of time of use is short, often times, shorter than the time required for maximum effectiveness, (ii) they are typically used infrequently at the time it matters most, which is after eating, drinking or snacking which are the times when the mouth is most susceptible to the potentially destructive effects of rapid and continuous formation of plaque and (iii) they are often inconvenient due to lack of portability.

As a result of the limitations with oral dentifrice, it is known to the art to develop oral care compositions, including confectionery compositions, which serve as vehicles for introducing oral care components to the mouth, particularly, to tooth surfaces.

In general, confectionery compositions have many advantages. They are convenient and portable. They are often used as vehicles to introduce and release components into the mouth and the body for many functions. The following are some common examples of oral health benefits that can be obtained from confectionery compositions: anti-cavity benefits conferred by chewing gum containing xylitol, the stimulation of salivary flow and palliation of dry mouth symptoms conferred by the sweetness of confectionery composition and by the act of chewing gum and the masking of bad breath (halitosis) conferred by using compositions such as breath mints and the like.

Furthermore, the formulations of confectionery compositions such as chewing gum, low boiled candy, hard boiled candy, pressed tablets, pressed mints, breath mints, coated candy, lozenges, pastes, throat drops and the like are well known in the art. These compositions are widely popular and well accepted among all age groups and where function is important, they can be present in the mouth for a longer duration of time than traditional oral dentifrices.

Today, consumers are increasingly desiring and demanding healthier, more natural and/or organic snacks and food products, preferably without an overload of sugar. Multi-vitamin and multi-mineral supplements and confectionery products are no exception to the demand. In response to the demand for orally healthy products, there are a variety of available products which are sugarless or sugar-free, which cater to dental health to some degree and/or which are made with natural and/or organic ingredients. These types of products do have their disadvantages however; one of which is that many of the sugarless and sugar-free products are sweetened with artificial sweeteners. Artificial sweeteners are generally known to be undesirable and harmful ingredients where long-term systemic health is concerned.

On the other hand, many available supplements and natural and/or organic confectionery products incorporate fermentable sugars. While fermentable sugars are added for sweetness, the daily consumption of sugary products is generally undesirable for good health; both systemic and oral. Whether refined, natural or organic, the source from which fermentable sugars are derived does not make a difference in terms of cariogenicity. Oral bacteria do not discriminate; these bacteria utilize any form of fermentable sugar to produce acid which is released into the oral environment, causing the demineralization of tooth structure and contributing to dental caries (decay or cavities). Thus, the sugar content and the frequent consumption of these products are known to be undesirable and harmful to dental health. Furthermore, as it relates to existing supplements for systemic health benefits, these compositions usually come in large tablet or capsule forms, which the many consumers find to be uncomfortable to swallow or take daily.

In summary, while there are many advantages associated with confectionery compositions in general, there are disadvantages associated with specific types of confectionery composition products that are available. This is because the regular consumption of products with fermentable sugar and/or artificial sweeteners are associated with poor oral health, disturbing side effects and many potential damages to long-term systemic health respectively.

Thus, it is apparent that the choices for confectionery products that cater to both oral and systemic health are limited. In view of the increasing demand for sugar-free, more natural, healthier and more nourishing food and snack products, there exists a need for confectionery compositions that not only cater to dental health but that also contribute to systemic health in a major, convenient and easy way.

Putting all these factors into consideration, the art is seeking portable products in the form of confectionery compositions which have anti-plaque components, are rich in nutrients and healthy components for dental and systemic health benefits respectively and which can be used or consumed safely, conveniently and comfortably throughout the day.

### SUMMARY OF INVENTION

The present invention relates to non-cariogenic, sugar-free, naturally sweetened confectionery compositions comprising: (i) a safe and effective amount of natural plant, tree and tea extracts selected from moringa, matcha green tea, green tea, chia and mixtures; extracts of which are rich in anti-oxidants, vitamins, minerals, essential amino acids and other nutrients and are beneficial to systemic and dental health; (ii) one or more anti-plaque component or components; (iii) one or more natural, non-cariogenic, tooth-friendly sweetener or mixtures of sweeteners; (iv) a suitable confectionery carrier material and (v) less than 10% water

Due to the inherent nature of soluble or chewable oral compositions such as tablets or gum products, prolonged contact with oral surfaces including teeth is achieved. This allows the anti-plaque components of the present invention to remain in contact with the teeth and other oral surfaces for an effective amount of time to help prevent or reduce plaque accumulation. The act of mastication also serves as a mechanical way of preventing or reducing plaque accumulation.

In addition, as the product remains soluble in the mouth and/or when the product is chewed, salivary flow is stimulated. Saliva that is stimulated and produced mixes with non-cariogenic sweeteners and natural plants or tree extracts containing anti-oxidants, vitamins, minerals, essential amino acids and other nutrients, thus allowing components to reach many areas of the oral surfaces to confer dental health benefits where applicable and/or to be swallowed into the body for systemic health benefits.

The insoluble portion of certain compositions such as the gum base portion of a chewing gum composition is not to be swallowed but rather, is to be used as a medium that allow anti-plaque components to be in contact with oral surfaces for a longer period of time for the occurrence of anti-plaque activities. Anti-plaque action here occurs by the constituents of the composition (non-cariogenic sweetener such as xylitol, and/or anti-oxidants present in certain extracts) and by the act of chewing the composition. The chewing action serves as a mechanical way of aiding in the prevention or reduction of plaque accumulation.

The delivery of these components in the form of tablets or chewing gum for example, insures that an adequate dosage of anti-plaque components and systemically beneficial nutrients are deliverable when the product is chewed and/or when the soluble portions are allowed to dissolve in the mouth and swallowed by the user. Furthermore, the sweet nature of the confectionery composition and the mere act of specifically chewing chewable confectionery compositions of the present invention encourages salivation, allowing saliva to wash out the mouth, help prevent cavities by its buffering action and provide lubrication which helps with the palliation of dry mouth symptoms.

The confectionery compositions of the present invention are convenient, portable and can be carried for consumption anytime and anywhere, throughout the day especially when away from home. Daily and consistent use of confectionery compositions of the present invention allow dental benefits that are comparable to those obtained by regular brushing with toothpaste or use of mouthrinse to be conferred effectively and allow additional safe and effective amount of natural plant extracts to be introduced into the body for systemic health benefits.

Generally, the overall health objective of the present invention is to use widely accepted, convenient and portable confectionery compositions to effectively confer a myriad of dental and systemic health benefits to the user/consumer.

When compared to products that are presently available, the present invention is unique in the following specific ways: (i) type of natural plants and/or tree extracts infused, which are selected from moringa, matcha green tea, green tea, chia and mixtures thereof; (ii) the variety of the combinations and mixtures of these natural plants and/or tree extracts and their components; iii) the mixtures of these extracts with one or more natural, non-cariogenic sweeteners for synergistic dental effects and (iv) the delivery of safe and effective amounts of nutrient-packed plants and/or tree extracts in compact, portable, convenient and sweet confectionery compositions for oral and systemic health benefits.

### DETAILED DESCRIPTION OF THE INVENTION

It should be understood that the detailed description and specific examples, while indicating the preferred embodiment of the invention, are intended for purposes of illustration only and are not intended to limit the scope of the invention.

### Definition of Terminology

The term “non-cariogenic” as defined herein means sweeteners which are not able to be metabolized by oral microbes and therefore do not contribute to the formation of dental caries.

The term “sugar-free” as defined herein means not containing fermentable refined, natural and/or organic sugars including but not limited to sucrose, honey, molasses, evaporated cane sugar, fructose, barley malt or rice syrup.

The term “natural” as defined herein means not artificial or containing artificial ingredients.

The term “anti-plaque” as defined herein means prevents or inhibits the accumulation of colonizing bacteria, bacterial deposits and food debris on the surfaces of the oral cavity such as on the surfaces of teeth.

The term “safe and effective amount” as used herein means an amount of a compound, component or composition sufficient to provide efficacious oral and systemic health benefits but low enough to avoid serious side effects within the scope of sound medical judgment.

The term “confectionery” as defined herein means a sweet, solid, gum, gum-like, or glassy composition containing non-cariogenic sweeteners. Examples of confectionery products include but are not limited to chewing gum, breath mints, low boiled candy, hard boiled candy, coated candy, lozenges, pastes, tablets, pressed mints, throat drops and the like; wherein these have suitable physical forms that include but are not limited to sticks, dragees, chicklets and batons.

The term “chewable confectionery composition” as used herein includes within its meaning chewing gum, and chewable and orally soluble tablets, troches and lozenges.

The term “chewing gum” as defined herein means a confectionery composition which is suitable for chewing. This includes bubble gum and confectionery products containing chewing gum, and coated chewing gum products.

Specific definitions of other special terms are provided in the category under which they are used.

Some ingredients may provide more than one dental and/or systemic health benefits and/or have more than one mode of action. Therefore, classifications made herein are for the sake of convenience and organization and are not intended to limit an ingredient to the particularly stated application or applications that are listed. The elements of the compositions, description of preferred embodiments and methods of manufacture of the present invention are described in more detail below.

### Natural Plants Extracts: Systemic and Dental Health Benefits

Moringa: Moringa is the sole genus in the flowering plant family Moringaceae, a multipurpose tree and is commonly known as “The Miracle Tree,” “Horseradish-tree,” or “Ben oil tree”. For the present invention, it is preferred that moringa extracts are derived from moringa oleifera, moringa stenopetala or mixtures thereof.

Moringa possesses a high nutritional value, provides many systemic health benefits and has a wide range of medicinal uses. It delivers anti-inflammatory support, promotes healthy digestion, increases mental clarity, increases energy, provides natural anti-aging benefits, nourishes body's immune system, regulates blood glucose levels and promotes healthy circulation as part of its many systemic health benefits.

Moringa contains many vitamins, minerals, vital proteins, antioxidants, omega oils, and other benefits which contribute to systemic health and directly or indirectly contribute to dental health. Specifically, it contains vitamin A (Alpha and Beta-Carotene), B, B1, B2, B3, B5, B6, B12, C, D, E, K, folate (Folic Acid), biotin, and contains minerals such as calcium, chloride, chromium, copper, fluorine, iron, manganese, magnesium, molybdenum, phosphorus, potassium, sodium, selenium, sulfur, zinc. Moringa has been shown to have four times the calcium of milk, three times the potassium of bananas, two times the protein of yogurt, four times the vitamin A of carrots, seven times the vitamin C of oranges. Furthermore, it contains all 9 essential amino acids (isoleucine, leucine, lysine, methionine, phenylalanine, threonine, tryptophan, valine, arginine) as well as the 9 additional amino acids (alanine, aspartic acid, cystine, glutamine, glycine, histidine, proline, serine, tyrosine) in addition to other beneficial ingredients which include chlorophyll, carotenoids, cytokinins, flavonoids, omega (3, 6, 9) oils, plant sterols, polyphenols, lutein, xanthins, rutin, and more.

For the present invention, it is preferred that the moringa extracts are in high quality forms which preserve as much of the plant nutrients as possible, in turn ensuring that as much systemic health components are conferred to the user/consumer as much as possible. The methods of extraction that preserve these desired and health beneficial plant components and properties are known to those well skilled in the art.

It is to be noted that the term “green tea” is used in two ways; first, it is used to broadly group all types of green tea which are derived from the leaves of a plant, Camellia sinensis and secondly, it is used specifically to differentiate regular green tea from matcha green tea because these two types of green tea are processed differently. The word “regular” is used as prefix to clarify the difference in meaning in certains sections where it is necessary to do so.

Green Tea: Green tea is made from the leaves of Camellia sinensis, the species of plant whose leaves and leaf buds are used to produce popular beverage teas. Teas such as white tea, yellow tea, green tea, oolong tea, black tea are all harvested from this species. However, the plant is processed differently to obtain each type of tea. For the present invention, it is most preferred that the tea extracts are obtained from green tea. It is also acceptable that other types of tea extracts may be included optionally.

Green tea is in a unique category in terms of the diverse ways it supports good health. It has been reported that almost every system of the body benefits from green tea. Green tea contributes to dental health by reducing plaque and fighting cavities and gum disease. Green tea also confers a myriad of systemic health benefits; it lowers cholesterol and blood pressure and prevents platelet aggregation (consequently reducing the risk of heart attack and stroke), is a cancer-fighter in that it inhibits the initiation, promotion, and progression of cancer thus helping to prevent cancer from starting and spreading. Green tea helps with diabetes in that it promotes normal blood sugar and insulin regulation. It bolsters immunity, has anti-microbial properties, fights infection by fighting against viruses and bacteria and is rich in fiber. It is a natural detoxifier; it helps lessen the effects of environmental toxins by aiding in the proper function of the liver which helps detoxify the body. It burns fat, burns calories and boosts metabolism. It improves poor mental function; promotes healthy cognitive functions, enhances mood and aids in concentration.

The most important active ingredients in green tea are polyphenols, caffeine, aromatic oils, nutrients (such as vitamin C, selenium, chromium, zinc and magnesium) and amino acid (such as L-Theanine). All beneficial health components of green tea are relevant to the present invention. However, polyphenols will be discussed further specifically for the dental health benefits and the caffeine and amino acid (L-theanine) contents of matcha green tea will be discussed further, specifically for their energy-boosting and calming benefits respectively.

Polyphenols present in green tea are also known as catechins. They are powerful antioxidant components of the green tea which are responsible for disease prevention and are active components of the treatment capability of green tea. The polyphenols in green tea are epigallocatechin gallate (EGCG), epigallocatechin (EGC), epicatechin gallate (ECG), epicatechin (EC), catechin (C). Of all the polyphenols, ECGC is believed to be the most powerful protector of dental health and the most powerful cavity fighter. Therefore, preferred tea extract mixtures are those with the most content of ECGC, though this is not intended to be limiting.

Matcha green tea: is a fine ground, powdered, high quality green tea that is reported to be much more potent than regular green tea by a ratio of about 1:10. It is distinct from green tea in that it is made from shade-grown tea leaves thus, its qualities and benefits are preserved closely to those of fresh green tea leaves. It is substantially richer in chlorophyll than other green teas, making it a superior daily detox in comparison. Matcha contains a high content of EGCG. It is reported that the concentration of the EGCG available from matcha is at least three times greater than the amount of EGCG available from other commercially available green teas. Matcha green tea is an energy booster due to its caffeine content and natural properties. The calming and relaxing effects are made possible by the rich content of a rare and unique amino acid, L-Theanine. It is believed to promote concentration and clarity of mind without any of the disadvantages or drawbacks of drinking coffee. In addition, L-Theanine in matcha green tea may help memory and learning ability while having an inhibitory effect on any possible side effects from the caffeine content. Therefore, for the present invention, non-limiting but preferred green tea extract mixtures where applicable, are those with higher matcha green tea content than regular green tea due to its added benefits and higher potency.

### Details of the Dental Benefits of Green Tea

Green tea helps prevent cavities, gum disease and resulting halitosis in several ways. First, green tea inhibits the growth of S. mutans and other bacteria associated with plaque. Secondly, green tea polyphenols reduce the risk of cavities by increasing the resistance of the tooth to the actions of cavity-causing bacteria and act in a synergist when fluoride is present. S. mutans produce enzymes such as glucosyltransferase which allow the bacteria to form larger areas of plaque on the tooth surface. Thirdly, polyphenols from green tea can lessen the risk of cavities by arresting the production of glucosyltransferase.

The qualities of an ideal anti-plaque agent have been reported to be an agent that eliminates disease-causing bacteria only, does not lead to the development of resistant bacteria, stays in the mouth for an extended period of time, is safe, reduces plaque and gingivitis, does not stain teeth, has no adverse effects on teeth, is easy to use and is inexpensive. Many available agents are lacking in at least one or more ways.

Green tea, however, is one of the very few agents that meets majority of the aforementioned qualities of an anti-plaque agent, possessing eight of the nine qualities; the exception being that it does not ordinarily stay in the mouth for an extended period of time.

The present invention aims to eliminate this exception in that it relates to a confectionery composition with a suitable carrier medium, such as a chewing gum composition, into which green tea is incorporated, thus allowing green tea and its beneficial components to be able to stay in the mouth for an extended period of time. In the present invention, green tea meets all the requirements of an ideal anti-plaque agent.

Furthermore, green tea is believed to have the potential to ease the pain of a toothache and may also prevent further damage if a root canal has become infected. The present invention presents a medium through which green tea may be gently pressed against an affected tooth for the purpose of alleviating pain. It also presents a safe medium by which the potential of green tea for preventing further damage from an infected root canal prior to being seen by a dentist may be assessed.

Chia: is also known as salvia hispanica. It is a species of flowering plant in the mint family, Lamiaceae. The nutritional benefits of chia include fiber, omega fatty acids, calcium, antioxidants, protein and more. Fiber is vital for all aspects of health, and is especially key for weight loss and digestion. In addition to the fiber content, it is reported that adding chia seeds to diet can reduce caloric intake and help lower the energy density (or calories) of foods. Chia seeds are said to deliver the maximum amount of nutrients with minimum calories. Chia is one of the most concentrated sources of omega-3 in any food. It also contains high amounts of omega-6. These essential fatty acids (EFAs) not only help build new cells and regulate various processes of the body but they support heart health and beautiful skin, hair and nails as well. It is important to obtain them from diet as they are not made in the body.

### Composition of Extracts and Preferred Embodiments

The confectionery composition may consist of one or more of natural plant or tree extract(s) in any of the following ways listed below. Herein, brackets (‘[ ]’) denotes a possible combination and mixture of natural plant or tree and associated extracts. The list is as follows: moringa, [moringa and matcha green tea], [moringa and green tea], [moringa and chia], [moringa, matcha green tea and green tea], [moringa, green tea and chia], [moringa, matcha green tea and chia], [moringa, matcha green tea, green tea and chia], matcha green tea, [matcha green tea and green tea], [matcha green tea and chia], [matcha green tea, green tea and chia], green tea, [green tea and chia], chia.

For the present invention, the following are most preferred embodiments: Generally:
(i) that all moringa extracts and inclusions are derived from moringa oleifera, moringa stenopetala or mixtures thereof.
(ii) that all green tea and matcha green tea extracts and inclusions are derived from camellia sinensis
(iii) that all chia extracts and inclusions are derived from salvia hispanica
(iv) that all natural plant or tree extracts and inclusions suitable for use in the present invention may be obtained from any part of the plant or tree including the leaf, stem, bark, pulp, flower, fruit, seed, flesh, juice, root or mixtures thereof
(v) that extracts for use in the present invention may be obtained by a variety of different methods including but not limited to pressing, extraction, distillation, or mixtures thereof; wherein the method or combination of methods are those that release extract components conferring the most amount of health benefits possible for the purpose of the present invention. These components include but are not limited to polyphenol (catechins), nutrients and minerals. The selection of the most effective method or methods are known to those well skilled in the art. Extracts for the present invention may be obtained from a wide variety of suppliers.
(vi) that as many ingredients as possible, including natural plant and tree extracts and sweeteners, are derived from natural, organic, non-genetically modified sources or mixtures thereof.
(vii) that the confectionery composition contains at least 0.0001% up to about 85% by weight of the composition, of natural plants or tree extracts
(viii) that ingredients and extracts may be incorporated in one or more physical forms; meaning that parts of natural plants or tree extracts and/or sweeteners and/or any other ingredients may be included as a whole form, as chopped or ground form, in powdery form, in oil form, in pure extracts form or other variation of extracts wherein only particular active components are isolated and included solely for the purpose of the present invention or mixtures of any of these forms thereof; wherein in for example, such a plant leaf may be included as whole leaf, as chopped or ground leaf, in powder form, in oil form (essential oil) or in pure extract form containing only polyphenols or a type of polyphenol such as ECGC for example, or in mixtures of two or more of these forms. The example given herein is non-limiting.

### Specific Preferences for Natural Plants or Tree Extracts

Moringa: For the present invention, it is preferred that moringa extracts are of high quality; quality grades that preserve as much of the plant nutrients as possible, in turn ensuring that as much of the beneficial systemic health components are conferred to the user/consumer as possible. The methods of extraction that preserve these desired and health beneficial plant components and properties are known to those well skilled in the art. However, to ensure the highest quality for the present invention, it is preferred that all moringa extracts are obtained from moringa trees that are organically raised without chemicals, harvested carefully and then, shade dried. This is reported to be the best way to ensure that essential nutrients remain intact.

For the compositions of the present invention, it is acceptable that any of the beneficial parts of the tree may be included, these are the leaf, stem, bark, pulp, flower, fruit, seed, flesh, juice, root or mixtures thereof. It is even more preferred that one or more of the leaf, flower, fruit, bark, seeds are included.

Green Tea and Matcha Green Tea: The active components in green tea and matcha green tea are believed to be the polyphenol catechines namely epigallocatechin gallate (EGCG), epigallocatechin (EGC), epicatechin gallate (ECG), epicatechin (EC) and catechin (C). Green tea and matcha green tea extracts suitable for use in the present invention are those that contain an effective amount of polyphenol catechins; wherein extracts suitable for use in compositions of the present invention preferably comprise greater than about 0.001%, preferably greater than about 5%, more preferably greater than about 10% and most preferably greater than about 15% up to 100%, by weight of the extract, polyphenol catechin material.

For the compositions of the present invention, higher quality and purer grades of green tea polyphenols which confer the most effective benefits are preferred over lower quality or less pure grades.

Chia: Chia extracts may be incorporated into the present invention as seeds or in powder form or in mixtures of these. For the purpose of this invention, it is understood that inclusion of chia may change the texture of certain compositions. However, it is preferred that the amount and/or the physical form of chia that is included in compositions of the present invention do not considerably change the texture much more than what would ordinarily be considered acceptable for that given composition. For example, it is preferred that the quantity of chia seeds or powder selected to be added to a chewing gum composition of the present invention does not considerably change the texture of the chewing gum composition beyond what would be considered acceptable.

Sweeteners: Sweeteners are included in the compositions of the present invention as a necessity for acceptable taste and additionally, for their non-cariogenic dental health benefits. Compositions of the present invention preferably comprise one or more non-cariogenic and natural sweetener(s); wherein the natural, non-cariogenic sweetener or mixtures of sweeteners is/are comprised of one or more polyols (sugar alcohols), one or more plant derived sweeteners or mixtures thereof.

It is preferred that polyol(s) are selected from the following: xylitol, erythritol, tagatose, isomaltutose, maltitol, mannitol, sorbitol, isomalt, glycerin or mixtures thereof. This list is however, not intended to be limiting.

Plant derived sweeteners may be included in the compositions of the present invention. The non-limiting representative example of such a plant derived sweetener is stevia. Stevia is preferred for its known sweetening and non-cariogenic nature. However, this specific preference is not intended to limit the inclusion of other plant-derived sweeteners.

It is preferred that stevia is derived from stevia leaf, specifically, stevia rebaudiana containing steviol glycoside (stevioside) and rebaudiana A; wherein it is most preferred that stevia extract is obtained from Rebaudiana A up to 98% or higher in purity and/or concentration, though non-limiting. Stevia may be included in one or more physical forms; for example, it may be included as whole leaf, ground leaf, as a powdered extract or oil or mixtures thereof.

Preferred embodiment of sweetener(s): Specifics given herein, with regards to grades, quality, purity or concentration of sweeteners are to indicate the most preferred embodiment, however, these specifics are not intended to be limiting as there are a variety of compositions considered. The selections of sweeteners may vary from composition to composition and/or from product to product. For the purpose of the present invention, it is to be noted that sweeteners may be obtained from a variety of suppliers.

Specific amounts and mixtures of sweeteners for acceptable taste and texture are well known to those skilled in the art. However, it is preferred that compositions of the present invention comprise greater than about 10%, preferably greater than about 20%, more preferably greater than about 30% and even more preferably greater than about 40% up to 99%, by weight of the composition, of non-cariogenic sweetener or mixtures of sweeteners.

Since xylitol is known for its dental benefits, for the compositions of the present invention, it is acceptable if xylitol is used as the only polyol for sweetening or included in mixtures of sweeteners whenever possible. It is preferred that the amounts of xylitol used in compositions of the present invention remain much lower than amounts that may typically produce undesirable laxative effects. While it is preferred that xylitol be included as a sweetener in any of the compositions of the present invention, it may be excluded if the non-cariogenic nature of the composition is still maintained.

No fermentable sugars or artificial sweeteners are to be included in any compositions of the present invention. Representative but non-limiting examples of such fermentable or artificial sweeteners are aspartame, cane sugar, acesulfame-K, saccharin, high fructose corn syrup, neotame, fructose, honey and the like.

Anti-plaque Components: For the present invention, anti-plaque activity is conferred from one or more polyol(s) such as xylitol, one or more natural plant extract(s) or mixtures thereof. For the purpose of the present invention, xylitol is the most preferred polyol because of its well known non-cariogenic property and similarity to sucrose in its appearance and sweetness. Furthermore, the inhibitory effects of xylitol on oral microbes are synergistic when used in conjunction with an extract since the extract is also acting to disable the microbes.

For the present invention, it is most preferred that the anti-plaque activities from a natural plant extract are obtained from matcha green tea, green tea or mixtures thereof. Furthermore, it is most preferred that the natural plant extract or mixtures of extracts contain an effective amount of polyphenol catechins; wherein the compositions of the present invention preferably comprise greater than about 1%, preferably greater than about 5%, more preferably greater than about 10% and most preferably greater than about 15% up to 100%, by weight of the extract, polyphenol material.

For the compositions of the present invention, it is acceptable that mixtures of tea (and associated extracts) may contain a higher amount of matcha green tea than regular green tea due to the additional benefits and higher potency associated with matcha green tea.

It is believed that, ECGC is the most powerful protector of dental health and the most powerful cavity fighter of all the polyphenols. For the anti-plaque purpose of the present invention and since matcha green tea has a higher content of ECGC, polyphenol extracts derived from matcha green tea and/or green tea may contain a higher content of ECGC than any other polyphenols whenever possible.

Additionally, compositions of the present invention may optionally include components that are believed to enhance the polyphenol efficacy and increase the stability of the polyphenol extract. These components may also independently confer other benefits including breath protection, anti-plaque protection and/or anti-bacterial efficacy.

Polyphenol extracts for use in the present invention can optionally be encapsulated. Such encapsulation may have several other benefits including stabilizing the polyphenols in the composition and can be present for providing controlled release mechanisms.

Encapsulation can be in the form of vesicles or lipsomes with uni-/bi- or multi-lamellar structures. These encapsulates can be formed by the use of emulsifying agents, fatty acids such as lecithin; they can be made using compounds that complex the polyphenols. Furthermore, polyphenols can be adsorbed within inorganic structures such as silica shell.

Additionally, compositions of the present invention may optionally include flouride ion sources for additional anti-cavity benefits.

Other Extracts and Additional Ingredients: Other tea extracts including but not limited to those selected from white tea, oolong tea and other beneficial extracts such as from yerba mate, ginger, turmeric, ginseng, cardamom, ashwagandha, licorice, echinacea, acai, lemongrass, garcinia cambogia, hibiscus may be added to any variations of mixtures of the present invention. Fruit extracts from fruits such as but not limited to cranberry, pomegranate may be included.

Flavoring: Compositions of the present invention can preferably comprise a flavoring agent and these agents are well known to those skilled in the art. Flavoring agent as used herein means those flavor essences and equivalent synthetic materials which add flavor to the composition. One or more flavoring agents may be included in the composition of the present invention and may also be included in one or more physical forms such as solid, liquid, essential oil, powder or encapsulated form or mixtures thereof.

For the present invention, flavors may be natural and/or synthetic flavors and may be present as oils and/or essences. It is most preferred if the flavors, oils or essences are derived from natural sources such as plants, roots, beans, nuts, leaves, flowers, fruits and so forth or mixtures thereof. Representative but non-limiting examples of suitable flavors include peppermint, menthol, spearmint, wintergreen, eucalyptus, lemon, lime, orange, banana, grape, vanilla, pomegranate, cranberry, apricot, grapefruit, apple, pear, strawberry, cherry, watermelon, birch, chocolate, anise, pineapple, coffee, cocoa, cola, peanut, almond, liquorice, mustard, cinnamon and the like.

The amount of flavoring agent employed is normally a matter of preference but in general they may be used in amounts up to about 5%, by weight of the composition. This amount is not intended to be limiting since preferred adjustments may be needed to maintain sweet taste in the presence of herbal ingredients to the degree that it is commonly acceptable.

Flavoring agents may also serve as warming or cooling agents for the present invention. A non-limiting example of preferred cooling agents is menthol which also includes menthol containing oils such as peppermint oil. Non-limiting examples of preferred warming agents are capsaicin, cinnamon oil.

Essential oils which are also known to have anti-microbial activity may be included in the compositions of the present invention. These oils include thymol, geraniol, carvacrol, hinokitiol, eucalyptol and mixtures thereof. A further group of natural extracts which add additional oral health benefits such as for control of halitosis may be included. These may be obtained from the plant groups consisting of but not limited to grape, grapefruit, orange, lemon, tangerine, bergamot mandarin, lime, or mixtures thereof. The compositions of the present invention may comprise from about 0.01% to about 5%, by weight of the composition, of antibacterial plant extract. These examples are not intendd to be limiting.

Additionally, ingredients or mixtures of ingredients which may be needed to optimize the delivery of all dental and systemic benefits and which may be needed to protect, preserve or prolong the integrity, esthetics, texture, taste, sensations and/or shelf-life of the product(s) of the present invention are well known to those skilled in the art and may be added.

Water: Water may or may not be included depending on the composition or product. It is preferred that the admixture used in the preparation of the chewable product such as chewing gum is significantly free of water. However, water used in the preparations where applicable, should preferably be of low ion content and free of organic impurities. The water content for compositions of this present invention is most preferably less than 2% up to less than about 10% by weight of the composition. Important considerations such as these are to prevent extracts from degrading or discoloring over time and also, so that the composition remains efficacious in delivering health benefits.

The preferred products forms of the compositions of the present invention are pressed tablets, low boiled candy, hard boiled candy and chewing gum since these are readily formulated with less than about 10%, by weight of the composition, water.

Confectionery Composition, Carrier Material and Additional Ingredients: Confectionery compositions of the present invention are sweet, solid, gum, gum-like, and/or glassy compositions containing non-cariogenic sweeteners. The confectionery compositions of the present invention are selected from chewing gum, low boiled candy, hard boiled candy, pressed tablets, pressed mints, breath mints, coated candy, lozenges, pastes, throat drops or mixtures thereof.

In general, there are several ingredients that are common to confectionery compositions; these include confectionery carrier materials, sweeteners, flavoring and the like.

Confectionery carrier material compositions for the present invention include chewing gum base, hard boiled candy base, low boiled candy base, gelatine base, compressed non-cariogenic sugar base or mixtures thereof. In addition, compositions of the present invention preferably comprise safe and effective levels of one or more of these additional ingredients which are well known to those skilled in the art. These additional ingredients are readily chosen based on the physical and esthetic properties desired for the compositions being prepared and/or based on properties that may help preserve, protect or prolong the effectiveness of oral health and systemic health care components of major ingredients.

Examples of such materials include, but are not limited to flavoring agents, fats, solvents, waxes (such as beeswax and carnauba wax), emulsifiers, plasticizing or softeners (such as lecithin), bulking agents, cationic material, buffers, whitening agents, alkali metal bicarbonate salts, calcium salts, thickening materials, humectants, lubricants, water, surfactants, titanium dioxide, flavoring agents, coloring agents, and mixtures thereof. Calcium salts may be incorporated as fillers and anti-cavity agents. Alkaline agents such as sodium bicarbonate may be incorporated to provide additional cleaning and breath-freshening properties.

The exact ingredients, types and amounts of ingredients for each product will differ from product to product and the techniques specific to each product are known to those well skilled in the art. For the present invention, it is most preferred that the types and amounts of these additional materials are selected such that the overall health objective of the present invention is maintained.

Chewable Confectionery Composition: For the purpose of the present invention, the term ‘chewable confectionery composition’ used herein generally denotes a subcategory of confectionery compositions which can be masticated for a given time. Additionally, these compositions may be fully soluble in the mouth or may be partially insoluble, having soluble components that can be released into the mouth. This chewable subcategory serves as an ideal confectionery prototype for accomplishing the purpose of the present invention, which is, to serve as a physically acceptable form that can optimally and effectively release active and/or nutritional components that confer benefits for dental and systemic health.

Chewable confectionery composition of the present invention includes chewing gum, chewable and orally soluble tablets, troches and lozenges. These chewable compositions are non-cariogenic and include candy, chewable and orally soluble tablets and chewing gum. Chewing gum includes bubble gum and confectionery products containing chewing gum, and coated chewing gum products.

Of all the listed confectionery compositions previously mentioned, chewing gum has the most functional advantage especially where dental health benefit is concerned. Chewing gum has masticatory properties wherein it can be chewed for the longest period of time as compared to other types of confectionery compositions, thus, providing additional mechanical benefits for oral health care in addition to the stimulation of salivary flow for the palliation of dry mouth symptoms.

For these unique advantages, chewing gum composition is used herein as a specific prototype and is discussed in further detail. It is to be understood, however, that the specific focus on chewing gum as a prototype is not intended to limit the scope of the present invention as there are variety of products included in the present invention that can accomplish the purpose of the invention. The following description of the preferred embodiment(s) is merely exemplary in nature and is in no way intended to limit the invention, its application, or uses.

Chewing Gum: General materials suitable for use in the practice of this invention and specifically, as it relates to chewing gums are well known to those skilled in the art. Chewing gum base for the present invention may be obtained from suppliers. Notwithstanding, several ingredients which are common to chewing gum compositions which are not readily used in other types of confectionery will be described in more detail. The specific descriptions of the components and list of materials for chewing gum compositions herein are not to intended to be limiting as such ingredients can be used in other types of confectionery if desired or needed.

Components of chewing gum: Chewing gum consists of gum base and one or more bulking agents and it may contain resins, fats and oils, waxes, softeners, and inorganic fillers. It also typically includes other components such as flavors, sensates and colors. While these ingredients are discussed herein, it is to be understood that gum base for chewing gum compositions are generally known to those well skilled in the art and therefore, for the purpose of the present invention, gum base for chewing gum compositions may be obtained entirely from suppliers.

The gum base is the water insoluble component of chewing gum that comprises a carrier material, or mixture of carrier materials, selected from elastomers, resins or waxes. They include natural or synthetic gum bases or mixtures thereof. Natural gums or elastomers include chicle, natural rubber, jelutong, balata, guttapercha, lechi caspi, sorva, guttakay, crown gum, perillo, or mixtures thereof. Synthetic gums or elastomers include butadiene-styrene copolymers, polyisobutylene and isobutylene-isoprene copolymers (butyl rubber) and styrene butadiene rubber. These examples are not intended to be limiting.

The gum base may comprise natural or synthetic ingredients or mixtures thereof. However, it is most preferred that gum base materials and combinations of materials are derived from as many natural sources as possible while preserving the non-cariogenic nature of the present invention and while maintaining the general scope and the purpose of the invention. This preference for natural ingredients over synthetic ingredients is not intended to be limiting especially since the gum base component of chewing gum composition is not soluble in the mouth nor is it to be ingested.

Elastomers are included to ensure that the elasticity, chewing texture and cohesiveness of the composition is acceptable. It is preferred that elastomer solvents are present where needed because they soften the elastomers. The selection of elastomers and elastomer solvents are well known to those skilled in the art.

The term “bulking agent” used herein means the non-cariogenic, water soluble component of a chewing gum not including flavors, high intensity sweeteners, colors, sensates, or encapsulated materials. The bulking agent functions to provide volume to the gum mass and to act to transport water soluble flavors, sweeteners and sensate agents at an acceptable rate in the mouth. For the present invention, it is preferred that the bulking agent comprise one or more non-cariogenic sweetener(s) such as xylitol.

For the present invention, the chewing gum bulking agents may optionally be of an improved kind, containing dietary fibers such as but not limited to inulin. The optional inclusion of dietary fiber(s) within the bulking agents is intended to be in addition to one or more non-cariogenic sweetener such xylitol, erythritol, tagatose, isomaltutose, maltitol, mannitol, sorbitol, isomalt. The selection and mixtures of these are well known to those skilled in the art.

In addition to natural and/or synthetic elastomers and bulking agents, insoluble gum base materials may contain resins, fats and oils, waxes, softeners, and inorganic fillers. The specific examples provided are not intended to be limiting. Resins may include polyvinylacetate and terpene resins and mixtures thereof. Fats and oils may be included in the gum base, including but not limited to vegetables oils, and cocoa butter. Waxes may include paraffin, beeswax, and carnauba and mixtures thereof. Softeners are also known as plasticizers or plasticizing agents and they are added to optimize the chewability and texture of the gum. Softeners that may be included in the present invention are glycerin, lecithin and combinations thereof. These examples are non-limiting as other softeners may be considered and included. Inorganic filler component may be included. Titanium dioxide may be used as a coloring agent.

Other ingredients and combinations of ingredients needed to preserve the integrity of product and optimize the delivery of all dental and systemic benefits are well known to those skilled in the art and may be added as needed.

### Embodiments of a Chewing Gum Composition

Gum base: It is preferred that the compositions comprise greater than about 10%, preferably greater than about 15%, more preferably greater than about 20% and most preferably greater than about 25% and up to about 75%, by weight of the composition, of gum base.

Elastomer: The gum base comprises a carrier material, or mixture of carrier materials, selected from elastomers, resins or waxes. Compositions of the present invention preferably comprise from about 2% to about 30%, more preferably from about 5% to about 25%, by weight, of elastomer.

Elastomer solvents: are also preferably present in compositions of the present invention since they aid softening of the elastomer component. Suitable solvents are known to those well skilled in the art. Compositions of a chewing gum of the present invention may comprise from about 2% to about 50%, by weight, of elastomer solvent.

Resins and Waxes: Resins are an optional, but desirable, ingredient of chewing gum compositions herein. They serve to plasticise the gum base. Suitable resins are well known to those skilled in the art. Such compositions may comprise from about 3% to about 25%, by weight, of resin.

The chewing gum compositions may also include one or more waxes. Suitable waxes are well known to those skilled in the art. Compositions may comprise up to about 25%, preferably from about 3% to about 20%, by weight, of wax.

Table 1 and 2 show examples of gum formulations. These examples along with the method of manufacture of chewing gum compositions described below are given solely for the purpose of illustrating the embodiments and method of manufacture within the scope of the present invention. These examples are not to be construed as limitations of the present invention as many variations of the invention are possible without departing from its objectives, purpose, aim or scope. Unless otherwise indicated, all ingredients are expressed as a weight percentage of the composition.

TABLE 1
Chewing Gum Formulation, Example 1

| Ingredients | Wt % |
|---|---|
| Xylitol | 35 |
| Gum Base | 30 |
| Extracts from Moringa, Matcha Green Tea, Green Tea) | 23 |
| Stevia | 5.0 |
| Natural Flavors | 2.2 |
| Lecithin | 2 |
| Gum Arabic | 1.5 |
| Carnauba wax (plant source natural glazing agent) | 1 |
| Titanium Dioxide (coloring agent) | 0.3 |
| Total | 100 |

TABLE 2
Chewing Gum Formulation, Example 2

| Ingredients | Wt % |
|---|---|
| Xylitol | 35 |
| Gum Base | 30 |
| Extracts from Moringa, Matcha Green Tea, Green Tea, Chia) | 25 |
| Gum Arabic | 2.5 |
| Natural Flavors | 2.2 |
| Lecithin | 1.9 |
| Resinous glaze | 1.4 |
| Beeswax | 0.9 |
| Carnauba wax (plant source natural glazing agent) | 0.8 |
| Titanium Dioxide (coloring agent) | 0.3 |
| Total | 100 |

Method of Manufacture: Techniques for formulations, equipment and processing for preparing and packaging chewing gum, candy, soluble and orally chewable tablets and lozenges are already well developed in the art. Therefore, it is preferred that the compositions of the present invention are prepared by the standard techniques that are well established and well known to those skilled in the art. When deemed necessary to those well skilled in the arts, however, improved techniques may be used to preserve the quality of extracts and ingredients and to prolong the general integrity of the compositions of the present invention.

The following examples further illustrate the preferred embodiments within the scope of the present invention. These examples of procedures are given for the purpose of illustration only. They are not to be understood as rigid or limiting in that the scope of the present invention is preserved even with many variations of the invention and procedural considerations. All ideal conditions, such as temperature, that are appropriate for making each type of compositions are well known to those skilled in the arts. For the sake of illustration, the procedure for formulating the chewing gum composition may be as follows:
(i) The gum base is melted
(ii) Effective amount(s) of one, more or all extracts and/or seeds is/are added
(iii) One or more of the sweeteners are added to the gum base to acceptable taste
(iv) One or more flavors are added
(v) A plasticizer is added
(vi) All ingredients are mixed for a sufficient period of time to ensure adequate dispersion.
(vii) The mixture is allowed to cool and is cut into suitable serving sizes.
(viii) For compositions comprising more than one phase: the different phases will be prepared separately and materials of similar phase will be partitioned and phases may be added in any order.
(ix) The two or more phases are combined with vigorous stirring resulting in a multiphase system eg an emulsion or dispersion. Considerations may be made for any ingredients which may be volatile or susceptible to generally breakdown at high temperatures. These ingredients may be added with gentle stirring after mixing of the different phases.
(x) Generally, coating is placed as a final step which may be applied by panning or spray dried and/or any techniques commonly known to those skilled in the art
(xi) It is preferred that the finished product is packaged in a way that air and moisture exposure is minimized. This is to enhance shelf stability.

What is claimed is:

1. A confectionery composition comprising: i) an effective amount of natural plants or tree extracts and mixtures thereof; ii) one or more anti-plaque agent(s); iii) one or more natural, non-cariogenic, tooth-friendly sweetener(s); iv) a suitable confectionery carrier material; and v) less than 10% water.

2. A composition according to claim 1 wherein the suitable confectionery carrier material is selected from chewing gum base, hard boiled candy base, low boiled candy base, gelatine base, compressed non-cariogenic sugar base and mixtures thereof.

3. A confectionery composition according to claim 2 wherein the confectionery composition is selected from chewing gum, low boiled candy, hard boiled candy, pressed tablets, pressed mints, breath mints, coated candy, lozenges, pastes, throat drops and mixtures thereof.

4. A confectionery composition according to claim 1 wherein one or more anti-plaque agent(s) are obtained from one or more polyol(s)/sugar alcohol(s), one or more extracts and mixtures thereof.

5. A confectionery composition according to claim 4 wherein the extract for anti-plaque purposes is obtained from the polyphenol (catechins) components of matcha green tea, green tea and mixtures thereof.

6. A confectionery composition according to claim 5 wherein the extract or mixtures thereof contain an effective amount of polyphenol (catechins); wherein extracts suitable for use in compositions of the present invention preferably comprise greater than about 1%, preferably greater than about 5%, more preferably greater than about 10% and most preferably greater than about 15% up to 100%, by weight of the extract, polyphenol (catechin) material.

7. A confectionery composition according to claim 4 wherein xylitol is used as the only polyol or wherein xylitol is one of the polyol included in any mixtures of sweeteners in the composition.

8. A confectionery composition according to claim 1 wherein the natural plant or tree extract or extracts is/are obtained from any part of the plant or tree including the leaf, stem, bark, pulp, flower, fruit, seed, flesh, juice, root and mixtures thereof.

9. A confectionery composition according to claim 1 wherein the composition contains 0.0001% up to 85% by weight of the composition, of extract.

10. The confectionery composition according to claim 8 wherein the natural plant or tree extract consists of moringa.

11. The confectionery composition according to claim 8 wherein the natural plant(s) or tree extracts consists of moringa and matcha green tea.

12. The confectionery composition according to claim 8 wherein the natural plant(s) or tree extracts consists of moringa and green tea.

13. The confectionery composition according to claim 8 wherein the natural plant(s) or tree extracts consists of moringa and chia.

14. The confectionery composition according to claim 8 wherein the natural plant(s) or tree extracts consists of moringa, matcha green tea and green tea.

15. The confectionery composition according of claim 8 wherein the natural plant or tree extract consists of moringa, matcha green tea and chia.

16. The confectionery composition according to claim 8 wherein the natural plant(s) or tree extracts consists of moringa, green tea and chia.

17. The confectionery composition according to claim 8 wherein the natural plant(s) or tree extracts consists of moringa, matcha green tea, green tea and chia.

18. A confectionery composition according to claim 1 wherein the natural, non-cariogenic sweetener or sweeteners is/are comprised of one or more polyols (sugar alcohols), one or more plant derived sweeteners or mixtures thereof.

19. A confectionery composition according to claim 22 wherein the composition contains greater than about 10%, preferably greater than about 20%, more preferably greater than about 30% and even more preferably greater than about 40% up to 99%, by weight of the composition, of non-cariogenic sweetener or sweeteners.

20. A confectionery composition according to claim 1 that includes pressed tablets, low boiled candy, hard boiled candy and chewing gum which are readily formulated with less than about 10%, by weight of the composition, water; wherein the confectionery composition contains less than about 10%, preferably less than about 8%, more preferably less than about 5%, even more preferably less than about 3%, and most preferably less than about 2%, by weight of the composition, water.
